(19) United States
(12) Patent Application Publication
(10) Pub. No.: US 2013/0261522 A1
(43) Pub. Date: Oct. 3, 2013

(54) BACK BRACE DEVICE

(71) Applicant: Debra BILLINGS, Perry, NY (US)

(72) Inventor: Debra BILLINGS, Perry, NY (US)

(21) Appl. No.: 13/438,737

(22) Filed: Apr. 3, 2012

(51) Int. Cl. A61F 5/00

(52) U.S. Cl. 602/19

(57) ABSTRACT

The present invention is a back brace device that includes a rectangular shaped pad that has two ends, a front facing, a back facing with a lower portion that are made of a foam material with a covering, a pair of securing straps that are attached to the two ends of the pad, wherein the pair of securing straps each have a distal end that have hook and loop material disposed on the distal end of the securing straps and a hook and loop strip that is disposed across the lower back portion of the back facing of the back brace device and forms a releasable hook and loop fastener with the hook and loop material disposed on the distal ends of the securing straps anywhere along the hook and loop strip to fittingly secure the back brace device to the user.

### TECHNICAL FIELD & BACKGROUND

An individual who suffers from pain from their vertebral column due to a medical procedure, may find it difficult to lean or rest against a chair or a sofa. It can be difficult for a person also with back problems to be at ease in a position that involves sitting for an extended period of time.

The present invention generally relates to a brace device. More specifically, the invention is a back brace device.

It is an object of the invention to provide a back brace device that provides comfort and support for a user that is recovering from back surgery or other similar surgery.

It is an object of the invention to provide a back brace device that reduces pressure applied to a user's back area.

It is an object of the invention to provide a back brace device that can be secured to a user at a plurality of locations along the user's back.

What is really needed is a back brace device that provides comfort and support for a user that is recovering from back surgery or other similar surgery that reduces pressure applied to the user's back area that can be secured to the user at a plurality of locations along the user's back.

### BRIEF DESCRIPTION OF THE DRAWINGS

The present invention will be described by way of exemplary embodiments, but not limitations, illustrated in the accompanying drawings in which like references denote similar elements, and in which:

FIG. 1A illustrates a front view of a back brace device, according to an embodiment of the present invention.

FIG. 1B illustrates a rear view of a back brace device, according to an embodiment of the present invention.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention. However, the order of description should not be construed as to imply that these operations are necessarily order dependent. In particular, these operations need not be performed in the order of presentation.

The phrase “in one embodiment” is utilized repeatedly. The phrase generally does not refer to the same embodiment, however, it may. The terms “comprising”, “having” and “including” are synonymous, unless the context dictates otherwise.

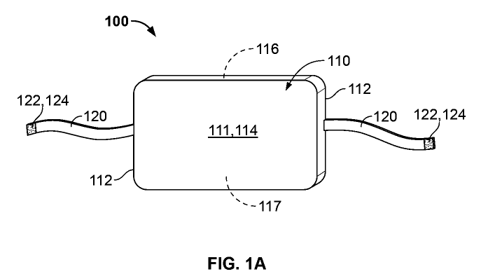

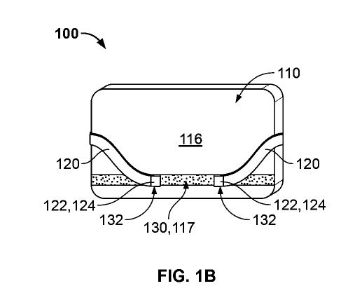

FIG. 1A illustrates a front view of a back brace device 100, according to an embodiment of the present invention. The back brace device 100 provides comfort and support for a user that is recovering from back surgery or other similar surgery that reduces pressure applied to the user's back area that can be secured to the user at a plurality of locations along the user's back.

The back brace device 100 includes a rectangular shaped pad 110 and a plurality of securing straps 120. The rectangular shaped pad 110 has two ends 112, a front facing 114, a back facing 116 with a lower portion 117 that are made of a foam material such as polyurethane, a polyurethane derivative or other suitable foam material. FIG. 1A illustrates the front facing 114 of the back brace device 100. The rectangular shaped pad 110 has a covering 111 such as fleece, fur or other suitable covering. The rectangular shaped pad 110 is approximately 5 inches in thickness but can be other suitable thicknesses as well. There are a plurality of securing straps 120 that can be a pair of securing straps 120 illustrated in FIG. 1A that are attached to the two ends 112 of the rectangular shaped pad 110 where one securing strap 120 is permanently attached to one end 112 and another securing strap 120 is permanently attached to the other end 112 of the rectangular shaped pad 110. The plurality of securing straps 120 can be any suitable number such as 4 or 6 securing straps. The pair of securing straps 120 each has a distal end 122 that have hook and loop material 124 disposed on the distal end 122 of the securing straps 120.

FIG. 1B illustrates a rear view of a back brace device 100, according to an embodiment of the present invention. The back brace device 100 has similar features to the back brace device 100 illustrated in FIG. 1A that include a rectangular shaped pad 110 and a pair of securing straps 120. The back brace device 100 illustrated and described in FIG. 1B and its description illustrates the back facing 116 of the back brace device 100. The back brace device 100 additionally includes a hook and loop strip 130.

The hook and loop strip 130 is disposed across the entire lower back portion 117 of the back facing 116 of the back brace device 100 and forms a releasable hook and loop fastener 132 with the hook and loop material 124 disposed on the distal ends 122 of the securing straps 120 anywhere along the hook and loop strip 130 to fittingly secure the back brace device 100 to the user. The hook and loop strip 130 and the hook and loop material 124 disposed on the distal ends 122 of the securing straps 120 allows the back brace device 100 to accommodate and fit a relatively large variety of different sized users that include relatively very small users to relatively very large users.

The back brace device permits a user to comfortably sit or lean without adding weight or pressure to their spine. The back brace device protects and supports a user's body from their head to their tail bone and is made of an approximate 5 inch thick pad typically made of foam material such as polyurethane or a polyurethane derivative which is covered in fleece or other suitable soft material. The pad is attached by two or more hook and loop fasteners and can be secured along a hook and loop strip disposed on the back of the pad to allow for the one or more straps to be secured anywhere along the strip. Chiropractors, doctors and their patients are among the many people who may find the back brace device relatively convenient and easy to use. The back brace device features two or more straps which can be placed in a crisscross or a straight orientation to make sure that the user is comfortable. The device is designed with an outer coating that is removable to facilitate ease of access for cleaning.

While the present invention has been related in terms of the foregoing embodiments, those skilled in the art will recognize that the invention is not limited to the embodiments described. The present invention can be practiced with modification and alteration within the spirit and scope of the appended claims. Thus, the description is to be regarded as illustrative instead of restrictive on the present invention.

1. A back brace device, comprising:
a rectangular shaped pad that has two ends, a front facing, a back facing with a lower portion that are made of a foam material with a covering;
a plurality of securing straps that are attached to said two ends of said rectangular shaped pad, wherein said securing straps each have a distal end that have hook and loop material disposed on said distal end of said securing straps; and
a hook and loop strip that is disposed across said lower back portion of said back facing of said back brace device and forms a releasable hook and loop fastener with said hook and loop material disposed on said distal ends of said securing straps anywhere along said hook and loop strip to fittingly secure the back brace device to said user.

2. The back brace device according to claim 1, wherein said rectangular shaped pad is made of polyurethane.

3. The back brace device according to claim 2, wherein said rectangular shaped pad is made of polyurethane derivative.

4. The back brace device according to claim 1, wherein said covering is made of fur.

5. The back brace device according to claim 1, wherein said covering is made of fleece.

6. The back brace device according to claim 1, wherein said rectangular shaped pad is approximately 5 inches in thickness.

7. The back brace device according to claim 1, wherein said securing straps include a pair of securing straps.

8. The back brace device according to claim 7, wherein one said securing strap is permanently attached to one said end and another said securing strap is permanently attached to said other end of said rectangular shaped pad.

9. The back brace device according to claim 1, wherein said hook and loop strip and said hook and loop material disposed on said distal ends of said securing straps allows said back brace device to accommodate and fit a large variety of different sized users.

10. The back brace device according to claim 9, wherein said large variety of users include a very small said user to a very large said user.

11. A back brace device, comprising:
a rectangular shaped pad that has two ends, a front facing, a back facing with a lower portion that are made of a foam material with a covering;
a pair of securing straps that are attached to said two ends of said rectangular shaped pad, wherein said pair of securing straps each have a distal end that have hook and loop material disposed on said distal end of said securing straps; and
a hook and loop strip that is disposed across said lower back portion of said back facing of said back brace device and forms a releasable hook and loop fastener with said hook and loop material disposed on said distal ends of said securing straps anywhere along said hook and loop strip to fittingly secure the back brace device to said user.

12. The back brace device according to claim 11, wherein said rectangular shaped pad is made of polyurethane.

13. The back brace device according to claim 12, wherein said rectangular shaped pad is made of polyurethane derivative.

14. The back brace device according to claim 11, wherein said outer coating is removable to facilitate ease of access to clean said outer coating.

15. The back brace device according to claim 11, wherein said covering is made of fur.

16. The back brace device according to claim 11, wherein said covering is made of fleece.

17. The back brace device according to claim 11, wherein said rectangular shaped pad is approximately 5 inches in thickness.

18. The back brace device according to claim 11, wherein said pair of straps which can be placed in a crisscross or a straight position.

19. The back brace device according to claim 11, wherein one said securing strap is permanently attached to one said end and another said securing strap is permanently attached to said other end of said rectangular shaped pad.

20. The back brace device according to claim 11, wherein said hook and loop strip and said hook and loop material disposed on said distal ends of said securing straps allows said back brace device to accommodate and fit a large variety of different sized users that include a very small said user to a very large said user.
